(19) United States
(12) Patent Application Publication
(10) Pub. No.: US 2015/0083627 A1
(43) Pub. Date: Mar. 26, 2015

(54) Gas Permeable Sterilization and Shipping Package System for Medical Products

(71) Applicant: Kimberly-Clark Worldwide, Inc., Neenah, WI (US)

(72) Inventor: Sean P. Gorman, Cumming, GA (US)

(73) Assignee: Kimberly-Clark Worldwide, Inc., Neenah, WI (US)

(21) Appl. No.: 14/037,573

(22) Filed: Sep. 26, 2013

(51) Int. Cl. A61B 19/02; B65D 51/16; B65D 65/40; B65D 41/32

(52) U.S. Cl. CPC A61B 19/026; B65D 41/32; B65D 51/1605; B65D 65/40
USPC 206/439; 53/425

(57) ABSTRACT

The present invention provides a gas permeable sterilization and shipping package system for medical products. The system includes a gas permeable sterilization package composed of an upper panel and a lower panel. Each panel has a main panel element, panel edges, and panel margins at or near the panel edges. The upper panel and the lower panel are joined at their respective panel margins so the respective main panel elements define a compartment for holding one or more articles to be sterilized. At least a portion of at least one panel is permeable to sterilant gas. The system includes at least one reinforcement strip joined to a panel at least at one location on its main panel element between its panel margins. Also disclosed is a method of sterilizing and shipping one or more items used in a medical procedure by utilizing the gas permeable sterilization and shipping package system.

### FIELD OF THE INVENTION

The present invention relates generally to a gas permeable bag which may be used in sterilization procedures, as well as other uses.

### BACKGROUND OF THE INVENTION

Sterilization of items used in medical procedures is important for reducing the spread of harmful and infectious agents to patients. Typically, the items are placed into a sterilization package or container such as sterilization wraps, vented rigid containers or into breathable pouches. Breathable pouches or bags are packaging for items used in medical procedures. These pouches or bags may be formed completely from a gas permeable material or may be composed of a gas permeable material joined to a gas impermeable material. These sterilization containers preserve sterility of the items contained therein, as well as the interior portion of these containers, after the containers and contents of the container have been through a sterilization procedure. In conventional processes, the gas permeable material allows a gas sterilant to enter the breathable bag, thereby allowing the gas sterilant to contact the item to be sterilized in the bag. Examples of current gas sterilization procedures include, gas plasma sterilization, steam sterilization, ethylene oxide sterilization, hydrogen peroxide sterilization, and ozone sterilization. Other sterilization procedures, such as irradiation have also been used.

In an alternative type of gas sterilization process, the items to be sterilized are placed within a container or bag and an amount of sterilant is injected into the container as it is sealed. Examples of such processes are described at, for example, U.S. Pat. No. 3,630,665 issued Dec. 28, 1971 to H. W. Anderson, et al. for “Method of Sterilization”; and U.S. Pat. No. 5,555,704 issued Sep. 17, 1996 to Caufield, et al. for “Sterilization System”. According to these patents, the bag or container may be evacuated to remove air prior to dispensing the sterilant into the bag or container. The sterilant may be a liquid that vaporizes into a sterilant gas. At least some portion of the bag or container should be permeable to the sterilant so that it dissipates through the bag or container at a predetermined rate that maintains sufficient sterilant in the bag or container to accomplish sterilization of its contents and, after a predetermined period, sufficiently depletes or dissipates the sterilant (preferably while held in a chamber to collect and safely disperse the sterilant) so it does not present a hazard to individuals opening the bag or container to access the sterilized items.

When such a gas sterilization procedure is used with a bag or flexible container such as a pouch that is evacuated, the injected sterilant typically does not provide sufficient gas volume to fully re-inflate the bag or pouch. Also, the volume of sterilant that dissipates through the bag or container after the predetermined period is typically not replaced by gas (e.g., air) infusing into the bag or container. As a result, the package has many projecting folds and corners resulting from the package being collapsed on itself and around the item or items to be sterilized. Such projecting folds or corners are a common source of failures or breaches in the integrity of the package. Such failures or breaches may be generated by shock, vibration and/or compression resulting from handling, stacking and shifting, or shipping of the package. Double wrapping or double packaging the items is not cost effective and may interfere with the proper dissipation of sterilant from the interior of the package. Reinforcing of the packaging may interfere with proper opening of the package and may also interfere with the proper dissipation of sterilant from the interior of the package.

Accordingly, there is a need for a gas permeable sterilization and shipping package system for medical products that is both practical and cost-effective at reducing breaches or failures in the integrity of the packaging. There is also a need for such a practical and cost-effective gas permeable sterilization and shipping package system for medical products that allows for easy and reliable opening of the package. This need also extends to a practical and cost-effective gas permeable sterilization and shipping package system for medical products that does not interfere with the proper dissipation of sterilant from the interior of the package.

### SUMMARY OF THE INVENTION

Generally stated, the present invention provides a gas permeable sterilization and shipping package system for medical products. The system is composed of a gas permeable sterilization package having an upper panel and a lower panel. Each panel has a main panel element, panel edges, and panel margins at or near the panel edges. The upper panel and the lower panel are joined at their respective panel margins so the respective main panel elements define a compartment for holding one or more articles to be sterilized.

The system also at least one reinforcement strip joined to a panel at least at one location on its respective main panel element between its panel margins. Desirably, the reinforcement strip is located on an exterior surface of a panel. Even more desirably, the reinforcement strip may be movable on the exterior surface of a panel between one or more locations where it is joined to the panel. The reinforcement strip may have a length such that it is a discrete element or strip that extends only between the panel margins or it may be longer. For example, the reinforcement strip may be a band extending completely around the package. The reinforcement strip may be a material selected from a polymeric film, a foil, paper, paper composite, fibrous webs, laminates thereof or combinations thereof.

In an aspect of the invention, the package may further include a frangible region on a main panel element at or adjacent at its respective panel margin. The frangible region is selected from the group consisting of score lines, thinned regions, lines of weakness, partial perforations, and the like. Alternatively and or additionally, the package may further include a closing device. Exemplary closing devices include tongue and groove sealing devices, adhesives and the like.

The upper panel and the lower panel may be independent sheets or may be formed from a single folded piece of material. Alternatively, the upper panel and the lower panel may be formed from a single length of extruded tube stock having sufficient diameter such that, when the tube stock is collapsed, the upper panel corresponds to the upper portion or layer of the tube stock and the lower panel corresponds to the lower portion or layer of the tube stock. At least a portion of at least one panel is a sterilant gas permeable material. The sterilant gas permeable material may be a material selected from a polymeric film, a nonwoven material, a paper material, and/or combinations thereof. In an aspect of the invention, the upper panel and the lower panel may each include a first edge and corresponding first margin; a second edge and second margin opposite from the first edge and first margin; a third edge and third margin extending between the first and second edges and margins, respectively; and a fourth edge and fourth margin opposite from the third edge and third margin and also extending between the first and second edges and margins, respectively. According to the invention, at least three margins of the upper panel may be joined to at least three margins of the lower panel.

The present invention encompasses a package for an item used in a medical procedure. The package includes a gas permeable sterilization package and shipping system as described above; at least one reinforcement strip joined to a panel at least at one location on its respective main panel element between its panel margins; and at least one item used in a medical procedure contained within the compartment defined by the main panel elements. The item used in a medical procedure may be one or more of a protective garment, a protective covering, a wound covering, a suture, a clamp, a scalpel, a retractor, forceps, scissors, a blade handle, a glove, a needle, a sponge, a syringe, a receptacle, a sealed vessel holding a therapeutic agent or combinations thereof. Alternatively and/or additionally, the item used in a medical procedure may be a medical procedure tray or kit containing a variety of items.

The present invention also encompasses a method of sterilizing and shipping one or more items used in a medical procedure. The method includes the steps of: providing a gas permeable sterilization and shipping package system for medical products as generally described above; inserting at least one item used in a medical procedure within the compartment defined by the main panel elements; evacuating air from the compartment and introducing a sterilant gas into the compartment; sealing the gas permeable sterilization and shipping package to contain the sterilant gas and the at least one item used in a medical procedure; holding the sealed gas permeable sterilization and shipping package to allow the sterilant gas to sterilize the at least one item and then to dissipate; packing the gas permeable sterilization package into a shipping container; and shipping the shipping container holding the gas permeable sterilization and shipping package containing the at least one item used in a medical procedure. The method may further include the step of wrapping the at least one item used in a medical procedure with a sterilization wrap prior to inserting it within the compartment defined by the main panel elements.

A better understanding of the above and many other features and advantages of the new gas permeable sterilization and shipping package system for medical products may be obtained from a consideration of the detailed description of the invention below, particularly if such consideration is made in conjunction with the appended drawings.

### BRIEF DESCRIPTION OF THE DRAWINGS

FIG. 1 shows a perspective view of an exemplary gas permeable sterilization package and shipping system.

FIG. 2 shows an exploded cross-section side view of an exemplary gas permeable sterilization package and shipping system.

FIG. 3. shows a cross-section side view of an exemplary gas permeable sterilization package and shipping system.

FIGS. 4A, 4B, 4C and 4D each show a front or top view of an exemplary an exemplary gas permeable sterilization package and shipping system illustrating alternative positions of the reinforcement strip(s).

FIG. 5 shows a perspective an exemplary gas permeable sterilization package and shipping system containing an item for a medical procedure.

FIG. 6 shows a perspective an exemplary gas permeable sterilization package and shipping system containing an item for a medical procedure partially opened.

FIG. 7 shows a perspective an exemplary gas permeable sterilization package and shipping system containing an item for a medical procedure more fully opened.

FIG. 8 shows a perspective view of an exemplary gas permeable sterilization package and shipping system incorporating an alternative reinforcement strip.

FIG. 9 shows a perspective view of an exemplary gas permeable sterilization package and shipping system highlighting details of an alternative reinforcement strip.

### DEFINITIONS

It should be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

As used herein, the term “sterilization” refers to techniques employed to attenuate, kill or eliminate harmful or infectious agents. Exemplary sterilization procedures include, for example, evacuating air from a container or bag containing an item to be sterilized, injecting a sterilant gas into the container or bag and sealing the container or bag. The sterilant gas may be ozone, hydrogen peroxide, ethylene oxide or the like. Exemplary sterilization techniques are described at, for example, U.S. Pat. No. 3,630,665 issued Dec. 28, 1971 to H. W. Anderson, et al. for “Method of Sterilization”; and U.S. Pat. No. 5,555,704 issued Sep. 17, 1996 to Caufield, et al. for “Sterilization System”, incorporated herein by reference.

The term “sterilizing conditions” refers to a combination of a concentration of sterilant and a time exposure interval which will sterilize an object which is subjected to a sterilant within a sterilizing container. Sterilizing conditions may be provided by a wide range of sterilant concentrations in combination with various time intervals. In general, the higher the concentration of a sterilant, the shorter a corresponding time interval is needed to establish sterilizing conditions. Accordingly, the effective amount of a sterilant may vary depending upon the length of exposure of the medical supplies to the sterilant.

As used herein, the term “sterilant gas permeable” is intended to mean a material which will allow sterilant gas to pass through the material but fails to allow airborne microbes, bacteria, viruses and mixtures thereof to pass through the material. Sterilant gas permeable materials are sometimes referred to in the art as selectively breathable materials.

As used herein, “gas impermeable” is intended to mean a material which does not readily allow gas to pass through the material. In addition, the gas impermeable material also fails to allow airborne microbes, bacteria, viruses and mixtures thereof to pass through the material.

As used herein, the term “opening element” refers to a mechanism or feature which facilitates the opening of the package by a user.

As used herein, the term “closing element” refers to a mechanism or feature which facilitates closing of the package by the user.

As used herein, the term “configure” or “configuration”, and derivatives thereof means to design, arrange, set up, or shape with a view to specific applications or uses. For example: a military vehicle that was configured for rough terrain; configured the computer by setting the system's parameters.

As used herein, the terms “substantial” or “substantially” refer to something which is done to a great extent or degree; a significant or great amount; for example, as used herein “substantially” as applied to “substantially” covered means that a thing is at least 70% covered.

As used herein, the terms “align,” “aligned,” and/or “alignment” refers to the spatial property possessed by an arrangement or position of things in a straight line.

As used herein, the terms “orientation” or “position” used interchangeably herein refer to the spatial property of a place where something is situated or a way in which something is situated; for example, “the position of the hands on the clock.”

As used herein, the term “about” adjacent to a stated number refers to an amount that is plus or minus ten (10) percent of the stated number.

### DETAILED DESCRIPTION OF THE INVENTION

The invention(s) disclosed herein relate generally to a gas permeable sterilization and shipping package system for medical products. More particularly, the invention(s) disclosed herein relate to a gas permeable sterilization and shipping package having at least one reinforcement strip joined to sterilization package at specified locations.

Reference will now be made in detail to one or more embodiments of the invention, examples of the invention, examples of which are illustrated in the drawings. Each example and embodiment is provided by way of explanation of the invention, and is not meant as a limitation of the invention. For example, features illustrated or described as part of one embodiment may be used with another embodiment to yield still a further embodiment. It is intended that the invention include these and other modifications and variations as coming within the scope and spirit of the invention.

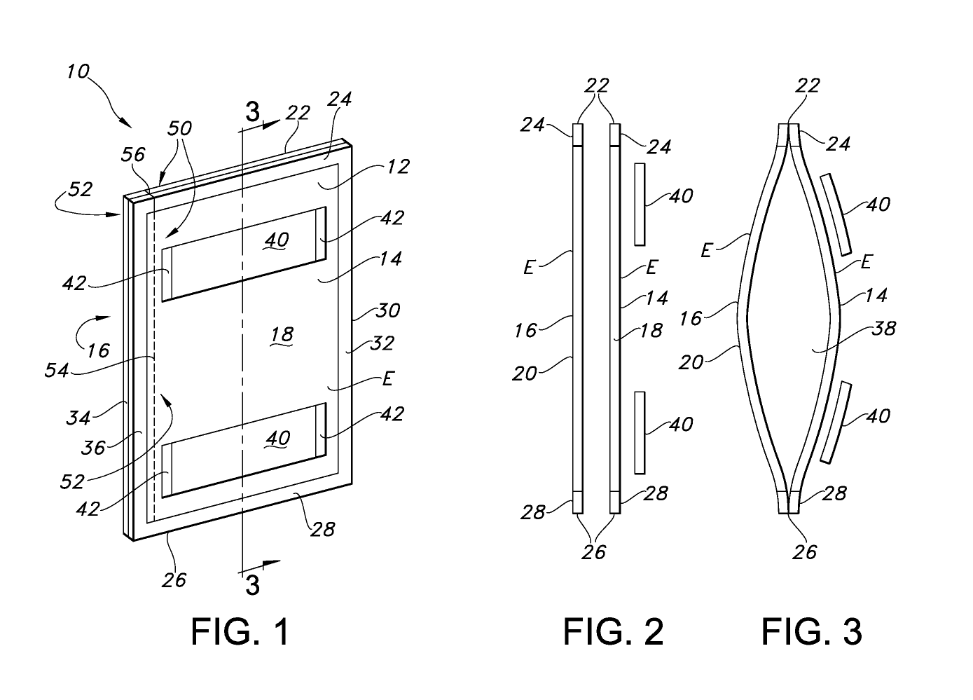

Turning now to the drawings, the invention is generally illustrated in perspective view at FIG. 1, in an exploded side cross-section view at FIG. 2 and in a side cross-section view at FIG. 3. The gas permeable sterilization and shipping package system 10 for medical products is composed of a gas permeable sterilization package 12 having an upper panel 14 and a lower panel 16. Each panel has a respective main panel element (main upper panel element 18 and main lower panel element 20), and exterior surface “E”, respective panel edges and panel margins at or near the panel edges. In this regard, the upper panel 14 and the lower panel 16 may each include a first edge 22 and corresponding first margin 24; a second edge 26 and second margin 28 opposite from the first edge 22 and first margin 42; a third edge 30 and third margin 32 extending between the first and second edges (22, 26) and margins (24, 28), respectively; and a fourth edge 34 and fourth margin 36 opposite from the third edge 30 and third margin 32 and also extending between the first and second edges (22, 26) and margins (24, 28), respectively. The upper panel 14 and the lower panel 16 are joined at their respective panel margins (24, 28, 32, and 36) so the respective main panel elements (18, 20) define a compartment 38 for holding one or more articles to be sterilized. The seal at the panel margins my range from about 1/16 inch to 1 inch (about 1.5 millimeters to about 25 millimeters) although it is contemplated that dimensions greater than 1 inch (25 millimeters) may be used. According to the invention, at least three margins of the upper panel may be joined to at least three margins of the lower panel to form a package which can then be sealed after an item for a medical procedure is inserted and sterilant gas is injected. The sterilization package 12 is illustrated as being rectangular but other configurations are such as a generally square, triangular, circular or the like are contemplated. The sterilization package can have a flat configuration as shown in FIG. 1. Other configurations are contemplated such as configurations that include additional panel(s) for three-dimensional configurations as well as configurations that include features such as, for example, pleats or corrugations. According to the invention, the upper panel 14 and the lower panel 16 may be independent sheets or may be formed from a single folded piece of material. When a single folded piece of material is used, the panel edge at the location of the fold may be characterized as the transition (e.g., fold line) between the upper panel 14 and the lower panel 16 when the single piece of material is folded and the upper panel 14 is collapsed onto the lower panel 16. The panel margin at the location of the fold may be characterized as the portions immediately adjacent the panel edge at the location of the fold. Alternatively, the upper panel and the lower panel may be formed from a single length of extruded tube stock having sufficient diameter such that, when the tube stock is collapsed, the upper panel 14 corresponds to the upper portion or layer of the tube stock and the lower panel 16 corresponds to the lower portion or layer of the tube stock. In such a configuration, the panel edges may be characterized as the transition (e.g., fold line) between the upper panel 14 and the lower panel 16 of the collapsed tube stock and the panel margins at these locations may be characterized as the portions immediately adjacent the panel edge at the location of the transition (e.g., fold line).

At least one of the upper panel 14 or the lower panel 16 has a portion containing a sterilant gas permeable material. This sterilant gas permeable material allows sterilant gas that is injected into the compartment 38 containing articles to be sterilized prior to sealing to pass to pass through the gas permeable material from the inside of the compartment to the outside of the sterilization package 12.

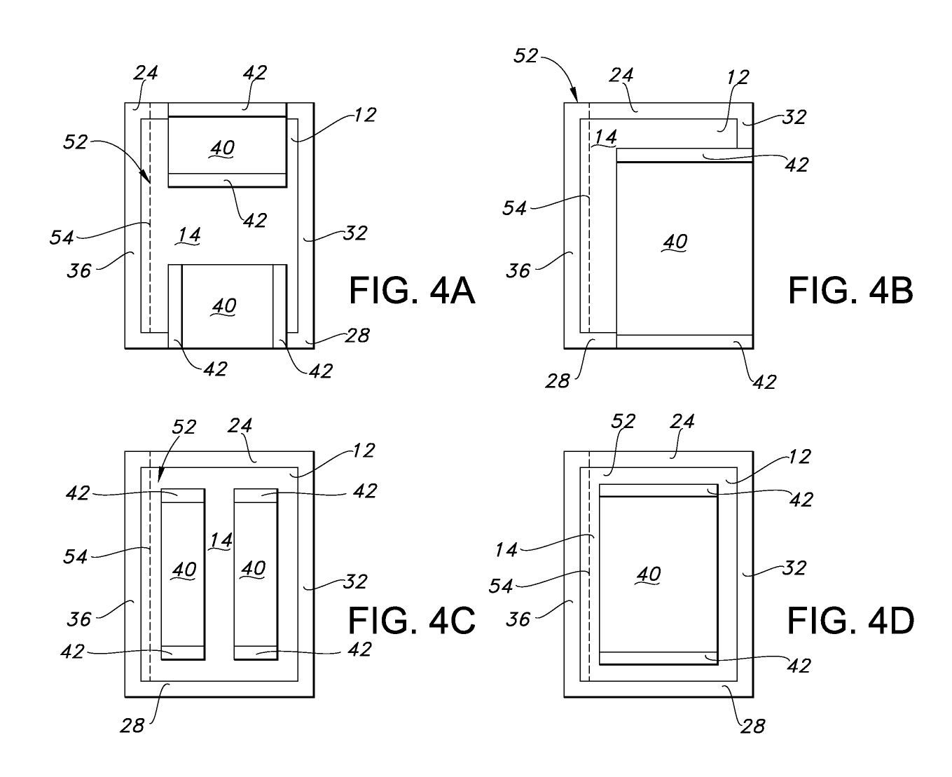

The gas permeable sterilization and shipping package system 10 for medical products also includes at least one reinforcement strip 40 joined to a panel (e.g., 14 or 16) at least at one location on its respective main panel element (e.g., 18 or 20) between its panel margins (e.g., 24, 28, 32). The reinforcement strip(s) is a desirably a discrete piece of material having a width, length and a thickness and which may be unwound from a roll or cut from a sheet. The reinforcement strip may be joined to the main panel element across an entire panel contacting surface of the reinforcement strip or may be joined to the main panel element at only discrete locations. The reinforcement strip may have a width ranging from less than 1 inch to greater than several inches, provided that its dimensions corresponds to a portion of the main panel element targeted for reinforcement (e.g., corresponds to the location(s) of one or more corner(s) or edge(s) of items contained in the package). FIG. 1 and FIGS. 4C and 4D illustrate examples in which the reinforcement strip(s) 40 is joined to a panel 14 between all of its panel margins (e.g., 24, 28, and 32). FIGS. 4A and 4B illustrate examples in which reinforcement strip(s) 40 is joined to a panel 14 between less than all of its panel margins. This feature is important in connection with sealing the opening to the compartment 38 to create the package 12 after one or more items is inserted into the compartment and sterilant gas is injected into the compartment. This feature is also important in connection with opening the package 12 to gain access to the items contained within the compartment 38. If the reinforcing strip 40 is not joined to the main panel element at a location between at least two panel margins, the reinforcing strip 40 may hinder or even prevent proper opening of the sterilization package 12.

Locating the reinforcing strips 40 over portions of the sterilization package 12 as describe above provides a gas permeable sterilization and shipping package system 10 that is particularly useful for gas sterilization processes in which items for a medical procedure are placed in a bag or flexible container such as a pouch which is then evacuated and a sterilant gas is injected as the bag or container is sealed to effect sterilization. In such processes, the injected sterilant typically does not provide sufficient gas volume to fully re-inflate the bag or pouch. Also, the volume of sterilant that dissipates through the bag or container after the predetermined period is typically not replaced by gas (e.g., air) infusing into the bag or container. As a result, the package has many projecting folds and corners resulting from the package being collapsed on itself and around the item or items to be sterilized.

Sterilization processes of this type are useful for individual trays or kits of medical items assembled for specific procedures. These trays or kits of items are packaged and sterilized at a central location and then shipped to hospitals, clinics, surgery centers or offices where they are opened and used. Shipping these trays or kits in conventional packages have revealed many modes of failure involving tears, cuts, punctures, holes or other breaches. Any failures may have serious consequences. The more common modes of failure were conventionally believed to involve tears, holes or cuts initiating from a medical procedure kit, tray or other content that was contained within the package. In other words, tears, cuts or holds were believed to begin at the interface between the sterilization tray or other content and the material of the package itself and propagate from the inside of the package penetrating outwardly through the material ultimately creating a breach. Accordingly, much effort has been expended to develop corner guards and other types of protection that is placed between the sterilization tray or other content and the package or bag.

While the inventor should not be held to any particular theory of operation, it has been discovered that pinholes and cuts most commonly propagate from the outside of a package (i.e., from friction or contact between the outside of a sterilization package and a shipping container) rather than propagating directly from contact or friction between the sterilization package and the sterilization tray or other content that is contained within the sterilization package. This is thought to be particularly applicable when the sterilization tray or other content is wrapped by or otherwise enclosed by conventional sterilization wrap fabric prior to being inserted into the compartment of the sterilization package because the sterilization wrap helps cushion the inside of the sterilization package from corners or edges of the contents or items that may cause pinholes and cuts. Accordingly, the applicant has discovered that locating the reinforcement strips 40 on the exterior or outward facing surface “E” of the main panel element(s) (e.g., 18 and/or 20) provides an unexpected advantage in preventing the formation of pinholes, tears and cuts during shipping of the sterilization package containing items for a medical procedure—particularly when the content of the sterilization package is wrapped or enveloped by sterilization wrap. Exemplary sterilization wrap and flexible sterilization assemblies are described by U.S. Pat. No. 5,958,337 issued Sep. 28, 1999 to Bourne et al. for “Single Step Sterilization Wrap System” and U.S. Pat. No. 8,261,963 issued Sep. 11, 2012 to Gaynor, et al. for “Flexible Multi-Panel Sterilization Assembly”, each incorporated herein by reference. Of course, the reinforcement strips 40 may be located only on an interior or inward facing surface of the main panel element(s) or on both the exterior or outward facing surface and the interior or inward facing surface of the main panel element(s).

According to an aspect of the invention, the reinforcement strip 40 may be movable on the exterior surface of a panel between one or more locations 42 where it is joined to the main panel element. That is, it is desirable for the reinforcement strip 40 to have some play or slack between the locations 42 so that the reinforcement strip may slide or shift to accommodate the shape of the contents in the compartment as well as in response to movement of the sterilization and shipping package system within a shipping container.

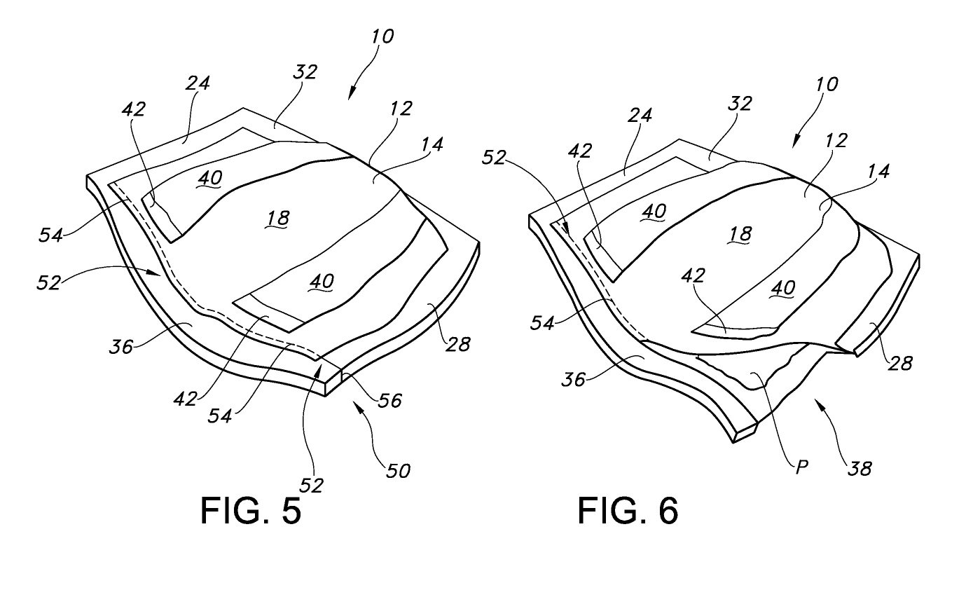

The sterilization package 12 of the present invention may also be provided with opening elements or opening features 50. For example, the sterilization package 12 may further include a frangible region 52 on a main panel element (e.g., 18 or 20) at or adjacent one or more of its respective panel margins (e.g., 24, 28, 32 and/or 36). The frangible region may be composed of one or more of score lines, thinned regions, lines of weakness, partial perforations, laser etching, notches, peelable seams, and the like. Combinations of these items are thought to be particularly useful. For example, referring to FIGS. 1, 4A through 4D, 5 and 6, the opening element or feature 50 is a frangible region 52 composed of partial perforations, scores, lines of weakness, etc. 54 (e.g., weakened portions that do not penetrate the main panel element 18 or compromise the effectiveness of the material as a barrier to preserve sterility) along and adjacent the fourth margin 36 as well as a notch 56 in the second margin 28. The notch 56 aids a user in tearing the second margin 28 composed of a material from both the upper panel 14 and the lower panel 16. The bonding of these two panels at the margin strengthens the pouch material in these areas, so it is desirable that the frangible region 52 extend through the panel margin so that the bonded panel margins can be easily torn through when opening the package to access the compartment is desired.

While the FIGS. 1, 4A through 4D, 5 and 6 illustrate the opening elements 50 adjacent only one panel margin, it should be understood that they may be may be located at or adjacent other panel margins. As can be seen from the drawings, these opening elements or features 50 should be located at or adjacent the panel margin and between the panel margin and the location when the reinforcing strip is joined to the main panel element. If the opening element is not located in these positions, the reinforcing strip may hinder proper opening of the sterilization package 12. FIGS. 1, 4A through 4D, 5 and 6 illustrate an opening element 50 is illustrated on only the upper panel 14, an opening element 50 may be used alternatively and/or additionally on the lower panel 16.

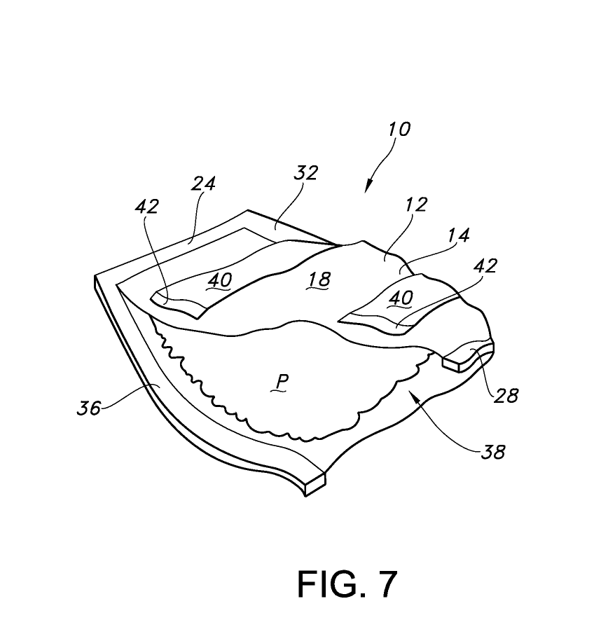

Referring to FIGS. 5, 6 and 7, there is illustrated in perspective view and exemplary sequence of steps to open the sterilization package with the frangible region 52 (e.g., a line of weakness 54). A user will typically start by grasping the sterilization package 12 near the panel margins (e.g., panel martins 28 and 36) and tear the sterilization package 12 along the line of weakness 54. As seen in these figures and in FIG. 1, a notch 56 or other starting point for the opening may also be provided. When the line of weakness 54 is torn by the user or person opening the sterilization package, access to the compartment 38 and the items “P” within the compartment is provided. The line of weakness 54 is illustrated in FIGS. 1, 4A-4D, 5 and 6 as a line parallel with the fourth panel margin 36 between that margin and the location 42 where the reinforcement strip 40 is joined to the main panel element 18. The line of weakness does not have to extend from one end of the package to the other edge of the package, but may stop short of the edge. Generally, it is desirable for ease of opening that the line of weakness extends to at least one of the edges. In addition, the line of weakness 54 or opening element 50 may located at or adjacent other panel margins.

Other opening elements or features 50 may also be used in the present invention. For example, pull tabs, pull flaps or other features on the upper and/or lower panels may be used to provide a user or person opening the sterilization package a place to grasp the sterilization package for opening.

In another embodiment of the present invention, the panel margins where the upper and lower panels are joined together may be peelable (e.g., with or without a chevron shape) to further facilitate opening of the sterilization package. For the panel margins to be peelable, the panel margins should readily separate from one another when subjected to a suitable tearing stress. Conventional peelable seals or seams are known to those skilled in the art and the methods of achieving peelable seals or seams are also known. For example, before joining the upper and lower panels together, one or more of the panel margins to be sealed may be provided with a release coating that will reduce seam strength. As a result, a person can use less force to open the sterilization package by breaking the seam or seal at the panel margin than would be required to peel the seam or seal apart if the release coating was not applied. Other method of creating a release seal include selecting adhesives which will tend to release when a tearing stress is applied. Examples of such adhesives include pressure sensitive adhesives. It is desirable that the seals or seams in the opening elements or features can be opened or broken with a sufficient but not excessive amount force. Determining the appropriate level of force is within the capability of one having ordinary skill in the art. If too much force is required to open the seam or seal, the sterilization package may open too rapidly due to the force needed, resulting in the items in the compartment being ejected from the sterilization package during opening.

Alternatively and or additionally, the package may further include a closing element or closing feature. While the margins of the sterilization package will usually be heat-sealed together and that the final margin after the items are inserted into the compartment will also be heat sealed, it is contemplated that closing elements or features such as, for example, conventional tongue and groove seals, adhesives and the like may be also be used. Generally speaking, any closing element may be used, provided the closing device can seal the sterilization package and prevent airborne microbes, bacteria, viruses and mixtures thereof from entering the sealed sterilization package. When an adhesive closing device is used, the adhesive may be protected with a peel strip which is removed before the sterilization package is sealed. The closing device of the present invention may also be configured to function as the opening device.

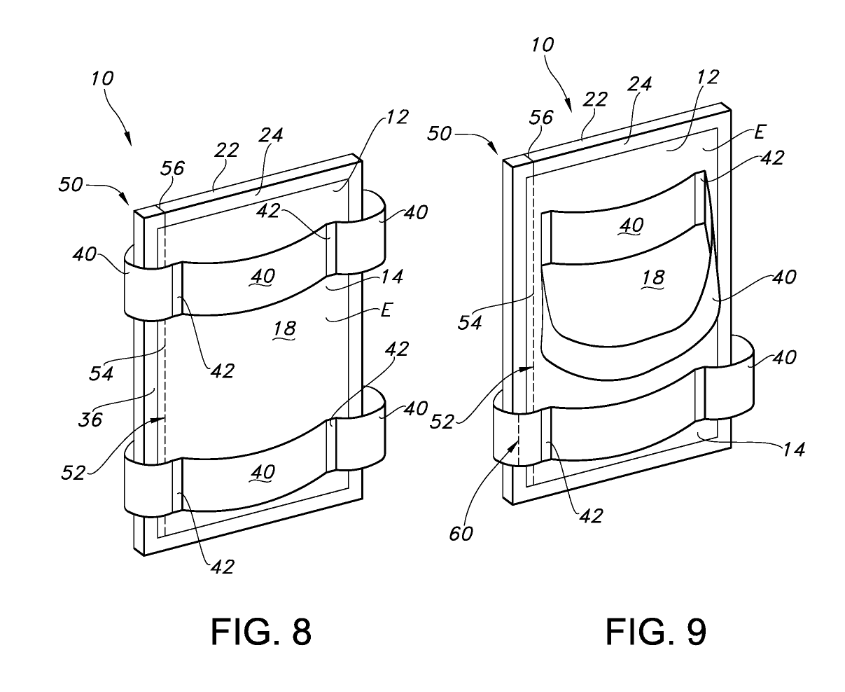

Referring to FIGS. 8 and 9, there is illustrated in perspective view an example of a gas permeable sterilization and shipping package system 10 for medical products that has another embodiment of the reinforcement strip 40. As can be seen in FIG. 8, the reinforcement strip 40 is joined to the main panel element 18 at locations 42 that are separated from the panel margins. The reinforcement strip 40 extends around the entire sterilization package 12. Referring to FIG. 9, one reinforcement strip 40 is illustrated in a configuration to allow for easy opening of the sterilization package. That is, the reinforcement strip 40 has been relocated from its position at the lower panel 16 and moved over the first panel edge 22 and first panel margin 24. This provides clear access to the opening element 50 in the form of a frangible region 52 provided by lines of weakness 54. The other reinforcement strip 40 illustrated in FIG. 9 as extending around the sterilization package 12 has an additional opening feature 60 which may be a frangible region provided by lines of weakness or the like. When such an additional opening feature 60 is utilized, the reinforcement strip 40 may be bonded or joined to the main panel elements of both the upper panel and the lower panel at locations that are separated from the panel margins.

In the present invention, suitable materials which may be used as the gas impermeable material include, for example, but are not limited to, polymeric plastic films, foils, paper, paper composites, fibrous webs and the like, laminates of one or more of these materials or a combination thereof of these materials. In addition, other materials which are not gas permeable may also be used. The gas impermeable material may be a single layer or a laminate of two or more layers.

Suitable materials can be polymeric materials including, but not limited to, polyethylene, polypropylene, polyester, nylon, and the like, as well as any combination thereof. Plastic film materials include, for example, a low density polyethylene (LDPE) film, a LDPE/LLDPE (linear low density polyethylene) film laminate, a LDPE/MDPE (medium density polyethylene) film laminate, a LDPE/HDPE (high density polyethylene) film laminate, an ethylene-vinyl alcohol (EVOH) or the like. In addition, films made from a polyethylene/polypropylene combination may also be used. Films incorporating metal coatings, also known as foils may also be used. In one embodiment of the present invention, the film materials used in the present invention include a polyolefin film, such as a polyethylene or polypropylene film. The thickness of the film can essentially be any thickness, provided that the film has sufficient strength that the articles contained within the compartment of the sterilization package do not puncture or otherwise compromise the film or the sterilization package.

It is also possible that the gas impermeable material is a laminate of a gas impermeable material and a gas permeable material. Examples of such laminates include, nonwoven/film laminates. These laminates may be beneficial to obtain a cloth-like feel to the outer or inner surface of the sterilization package or to reinforce or protect the film material from damage caused by the articles in the compartment of the sterilization package or from elements outside of the sterilization package.

Essentially any sterilant gas permeable material may be used in the present invention, provided that the material is permeable to a sterilizing gas but impermeable to airborne microbes, bacteria, viruses and mixtures thereof. Suitable gas permeable materials useable in the present invention include, for example, medical grade paper, nonwoven materials and other similar gas permeable materials.

Suitable nonwoven materials useable as the gas permeable material of the sterilization package of the present invention include, for example, airlaid nonwoven webs, spunbond nonwoven webs, meltblown nonwoven webs, bonded-carded-webs, hydroentangled nonwoven webs, spunlace webs and the like. The method of manufacturing each of these materials is known in the art. Laminates of these materials may also be used.

Of these nonwoven materials, the fibrous material web may comprise a nonwoven meltblown web. Meltblown fibers are formed by extruding a molten thermoplastic material through a plurality of fine, usually circular, die capillaries as molten fibers into converging high velocity gas (e.g. air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin, et al. Generally speaking, meltblown fibers may be microfibers that may be continuous or discontinuous, and are generally smaller than 10 microns in diameter, and are generally tacky when deposited onto a collecting surface.

The nonwoven material web may be a nonwoven spunbond web. Spunbonded fibers are small diameter substantially continuous fibers that are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spun-bonded nonwoven webs is described and illustrated, for example, in U.S. Pat. No. 4,340,563 to Appel, et al., U.S. Pat. No. 3,692,618 to Dorschner, et al., U.S. Pat. No. 3,802,817 to Matsuki et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Levy, U.S. Pat. No. 3,542,615 to Dobo, et al., and U.S. Pat. No. 5,382,400 to Pike, et al. Spunbond fibers are generally not tacky when they are deposited onto a collecting surface. Spunbond fibers can sometimes have diameters less than about 40 microns, and are often between about 5 to about 20 microns.

The nonwoven material web may also comprise a laminate material such as a spunbond/meltblown/spunbond, or SMS, material. A typical SMS material is described in U.S. Pat. No. 4,041,203 to Brock et al. Other SMS products and processes are described, for example, in U.S. Pat. No. 5,464,688 to Timmons et al.; U.S. Pat. No. 5,169,706 to Collier et al.; and U.S. Pat. No. 4,766,029 to Brock et al. Generally, an SMS material will consist of a meltblown web sandwiched between two exterior spunbond webs. Such SMS laminates have been available commercially for years from Kimberly-Clark Corporation under marks such as Kimguard®. The spunbonded layers on the SMS laminates provide durability and the internal meltblown layer provides porosity.

In another embodiment of the present invention, the permeable material may be a laminate of a film layer and a fibrous material layer which have been laminated together by any lamination technique known to those skilled in the art. Examples of these laminates include, for example, spunbond-film laminates (SF), and other such laminates. Again, it is necessary for the laminate to be gas permeable. In one embodiment, the material for the wrapper component are prepared from a film/spunbond laminate material available from Kimberly-Clark Corp, and known as HBSTL (“highly breathable stretch thermal laminate”), and which material is further disclosed in U.S. Pat. No. 6,276,032, the entire disclosure of which is hereby incorporated herein by reference.

Suitable lamination means which may be used to form the gas permeable laminate materials include, but are not limited to, adhesives, needle punching, ultrasonic bonding and thermomechanical bonding as through the use of heated calendering rolls. Such calendering rolls will often include a patterned roll and a smooth anvil roll, though both rolls may be patterned or smooth and one, both or none of the rolls may be heated. Calendering may also be used to place an aesthetic pattern defined in the laminated material.

Other materials for the gas permeable material and the gas impermeable materials may be used for their properties. For example, if it is necessary for the compartments to be expandable, elastic materials, including elastic webs and elastic nonwovens may also be used. Again, the only requirement is that the one of the panels is gas permeable.

Each of the upper and/or lower panels may have topical treatments applied thereto for more specialized functions. Such topical treatments and their methods of application are known in the art and include, for example, alcohol repellency treatments, anti-static treatments, non-slip treatments and the like, applied by spraying, printing, dipping, or other methods known to those skilled in the art. An example of such a topical treatment is the application of ZELEC® antistatic neutralized mixed alkyl phosphates (available from E.I. DuPont, Wilmington, Del.). Non-slip treatments are placed on the outside of the sterilization package which aid a user to grab the sterilization package and open it. Often users must open the sterilization packages wearing protective articles, such as gloves, when the sterilization package compartment contains items used in a medical procedure. Having gloves donned may make it difficult to open the sterilization package to retrieve the items contained within the compartment. Non-slip treatments usable in the present invention can be formed in a variety of manners including applying a coating to the entire surface of the sterilization package or applying a coating in discrete areas on the sterilization package. The coating should have a higher coefficient of friction than the material used to prepare the sterilization package. Examples of such non-slip treatments include, but are not limited to, placing an elastomeric material on at least a portion of the upper panel or the lower panel.

Other features which may be provided to the sterilization package of the present invention include, providing the sterilization package with a pre-printed or printable surface. The pre-printed surface would provide information or instructions about opening the sterilization package or the contents of the sterilization package. The printable surface would be useful for marking with a permanent marker to identify contents of the package or other notes, such as the time and date the item within the sterilization package was sterilized.

The present invention encompasses a package for an item used in a medical procedure. The package includes a gas permeable sterilization package and shipping system as described above; at least one reinforcement strip joined to a panel at least at one location on its respective main panel element between its panel margins; and at least one item used in a medical procedure contained within the compartment defined by the main panel elements. The item used in a medical procedure may be a medical procedure tray or kit and includes a one or more of a protective garment, a protective covering, a wound covering, a suture, a clamp, a scalpel, a retractor, forceps, scissors, a blade handle, a glove, a needle, a sponge, a syringe, a receptacle, a sealed vessel holding a therapeutic agent or combinations thereof. In addition other items used in medical procedures not specifically mentioned may be included. The sterilization package and shipping system of the present invention could also be used as a container for other various items, which will readily apparent to those skilled in the art.

The present invention also encompasses a method of sterilizing and shipping one or more items used in a medical procedure. The method includes the steps of: providing a gas permeable sterilization and shipping package system for medical products as generally described above; inserting at least one item used in a medical procedure within the compartment defined by the main panel elements; evacuating air from the compartment and introducing a sterilant gas into the compartment; sealing the gas permeable sterilization package to contain the sterilant gas and the at least one item used in a medical procedure; holding the sealed gas permeable sterilization package to allow the sterilant gas to sterilize the at least one item and then to dissipate; packing the gas permeable sterilization package into a shipping container; and shipping the shipping container holding the gas permeable sterilization and shipping package containing the at least one item used in a medical procedure. The method may further include the step of wrapping the at least one item used in a medical procedure with a sterilization wrap prior to inserting it within the compartment defined by the main panel elements. The shipping container may be any conventional shipping container such as a box, bin, crate, pouch, reinforced envelope or the like.

It is contemplated that the sterilization package and the items contained within the sterilization package may be sterilized by alternative means such as by conventional sterilization processes in which the items to be sterilized are first sealed within the sterilization package and then exposed to a sterilization gas in a sterilization chamber in a more conventional sterilization procedure. For example, the sterilization package with the items contained therein may be placed in a sterilization chamber to be exposed to, for example, gas plasma sterilization, steam sterilization, ethylene oxide sterilization, hydrogen peroxide sterilization, and ozone sterilization. While the sterilization package and shipping system of the present invention is designed for gas sterilization, this does not mean that other sterilization procedures, such as irradiation, cannot be used. It is believed that the sterilization package and shipping system of the present invention can be used in most sterilization procedures, provided that the materials from which the sterilization package and shipping system is prepared are stable in the sterilization procedure used.

Those skilled in the art will recognize that the present invention is capable of many modifications and variations without departing from the scope thereof. Accordingly, the detailed description and examples set forth above are meant to be illustrative only and are not intended to limit, in any manner, the scope of the invention as set forth in the appended claims.

1. A gas permeable sterilization and shipping package system for medical products, the system comprising:
a gas permeable sterilization package comprising an upper panel and a lower panel, each panel having:
a main panel element,
panel edges, and
panel margins at or near the panel edges,
wherein the upper panel and the lower panel are joined at their respective panel margins so the respective main panel elements define a compartment for holding one or more articles to be sterilized, at least a portion of at least one panel comprises a sterilant gas permeable material; and
at least one reinforcement strip joined to a panel at least at one location on its respective main panel element between its panel margins.

2. The package system of claim 1, wherein the upper panel and the lower panel are formed from a single folded piece of material.

3. The package system of claim 1, wherein the upper panel and the lower panel are formed from a single extruded length of tube stock.

4. The package system of claim 1, wherein package further comprises an opening element on a main panel element at or adjacent at its respective panel margin.

5. The package system of claim 4, wherein the opening element is a frangible region is selected from the group consisting of score lines, thinned regions, lines of weakness, partial perforations, laser etching, notches, peelable seams, and the like.

6. The package system of claim 1, wherein the sterilant gas permeable material comprises material selected from a polymeric film, a nonwoven material, a paper material, and/or combinations thereof.

7. The package system of claim 1, wherein the reinforcement strip comprises a material selected from a polymeric film, a foil, paper, paper composite, fibrous webs, laminates thereof or combinations thereof.

8. The package system of claim 1, wherein the reinforcement strip is located on an exterior surface of a panel.

9. The package system of claim 8, wherein the reinforcement strip is movable on the exterior surface of a panel between one or more locations where it is joined to the panel.

10. The package system of claim 1, further comprising a closing device.

11. The package system of claim 1, wherein the upper panel and the lower panel each include a first edge and corresponding first margin; a second edge and second margin opposite from the first edge and first margin; a third edge and third margin extending between the first and second edges and margins, respectively; and a fourth edge and fourth margin opposite from the third edge and third margin and also extending between the first and second edges and margins, respectively and further wherein at least three margins of the upper panel are joined to at least three margins of the lower panel.

12. The package system of claim 9, wherein the reinforcement strip is a band extending completely around the package.

13. A gas permeable sterilization and shipping package system for medical products, the system comprising:
a gas permeable sterilization package comprising an upper panel and a lower panel, each panel having:
a main panel element,
panel edges, and
panel margins at or near the panel edges,
wherein the upper panel and the lower panel are joined at their respective panel margins so the respective main panel elements define a compartment for holding one or more articles to be sterilized, at least a portion of at least one panel comprises a sterilant gas permeable material; and
at least one reinforcement strip located on an exterior surface of a panel, the reinforcement strip being joined to a panel at least at one location on its respective main panel element between its panel margins and movable on the exterior surface of the panel between the at least one location where it is joined to the panel.

14. The package system of claim 13, wherein the upper panel and the lower panel each include a first edge and corresponding first margin; a second edge and second margin opposite from the first edge and first margin; a third edge and third margin extending between the first and second edges and margins, respectively; and a fourth edge and fourth margin opposite from the third edge and third margin and also extending between the first and second edges and margins, respectively, and further wherein at least three margins of the upper panel are joined to at least three margins of the lower panel.

15. The package system of claim 13, wherein the reinforcement strip is a band extending completely around the package.

16. A package for an item used in a medical procedure, the package comprising:
a gas permeable sterilization package and shipping system comprising an upper panel and a lower panel, each panel having:
a main panel element,
panel edges, and
panel margins at or near the panel edges,
wherein the upper panel and the lower panel are joined at their respective panel margins so the respective main panel elements define a compartment for holding one or more articles to be sterilized, at least a portion of at least one panel comprises a sterilant gas permeable material;
at least one reinforcement strip joined to a panel at least at one location on its respective main panel element between its panel margins; and
at least one item used in a medical procedure contained within the compartment defined by the main panel elements.

17. The package of claim 16, wherein the item used in a medical procedure comprises a one or more of a protective garment, a protective covering, a wound covering, a suture, a clamp, a scalpel, a retractor, forceps, scissors, a blade handle, a glove, a needle, a sponge, a syringe, a receptacle, a sealed vessel holding a therapeutic agent, a medical procedure tray or kit, or combinations thereof.

18. A method of sterilizing and shipping one or more items used in a medical procedure, the method comprising:
providing a gas permeable sterilization and shipping package system for medical products, the system comprising:
a gas permeable sterilization package comprising an upper panel and a lower panel, each panel having:
a main panel element,
panel edges, and
panel margins at or near the panel edges,
wherein the upper panel and the lower panel are joined at their respective panel margins so the respective main panel elements define a compartment for holding one or more articles to be sterilized, at least a portion of at least one panel comprises a sterilant gas permeable material; and
at least one reinforcement strip joined to a panel at least at one location on its respective main panel element between its panel margins;
inserting at least one item used in a medical procedure within the compartment defined by the main panel elements;
evacuating air from the compartment and introducing a sterilant gas into the compartment;
sealing the gas permeable sterilization and shipping package to contain the sterilant gas and the at least one item used in a medical procedure;
holding the sealed gas permeable sterilization package to allow the sterilant gas to sterilize the at least one item and then to dissipate;
packing the gas permeable sterilization and shipping package into a shipping container; and
shipping the shipping container holding the gas permeable sterilization and shipping package containing the at least one item used in a medical procedure.

19. The method of claim 18, wherein the step of inserting at least one item used in a medical procedure within the compartment defined by the main panel elements further includes wrapping the at least one item with a sterilization wrap prior to inserting it with the compartment.

20. The method of claim 18, wherein the sterilant gas comprises ethylene oxide.
